(19) United States
(12) Patent Application Publication
(10) Pub. No.: US 2007/0237719 A1
(43) Pub. Date: Oct. 11, 2007

(54) METHOD AND SYSTEM FOR MONITORING AND ANALYZING COMPLIANCE WITH INTERNAL DOSING REGIMEN

(76) Inventors: Christopher M. Jones, Midland, MI (US); Peter K. Mercure, Midland, MI (US); Shakil Saghir, Midland, MI (US)

Correspondence Address: Thomas Moga;Butzel Long, STONERIDGE WEST, 41000 WOODWARD AVENUE, BLOOMFIELD HILLS, MI, 48304, US

(21) Appl. No.: 11/693,404

(22) Filed: Mar. 29, 2007

Related U.S. Application Data
(60) Provisional application No. 60/787,623, filed on Mar. 30, 2006.

(51) Int. Cl. A61K 49/00; G06Q 50/00

(52) U.S. Cl. 424/9.2; 424/9.6; 600/300; 705/3

(57) ABSTRACT

A method and system for monitoring and analyzing compliance with an internal dosing regimen prescribed to be taken in multiple dose forms includes the steps of detecting internalization of a first dose form to generate a first data point, detecting internalization of a second dose form to generate a second data point, and analyzing the first data point and the second data point. The step of analyzing the first and second data points generates a metric of a variety of possible metric types. The first and second dose forms may be two of any plural number of sequentially-internalized dose forms which generate a like number of sequential data points. Subsequent internalizations of dose forms result in at least a like number of data points being generated. To effect the disclosed method a system is provided which includes at least two dose forms, a time stamp identifier operatively associated with each dose form, a receiving device for receiving the time stamp identifier data, and an analyzer for analyzing the received data,

### CROSS-REFERENCE TO RELATED APPLICATIONS

This application claims the benefit of U.S. Provisional Patent Application No. 60/787,623, filed Mar. 30, 2006.

### BACKGROUND OF THE INVENTION

The present invention relates to a method and system for monitoring compliance to an internal dosing regimen and the subsequent analysis of the data generated. More particularly, the present invention relates to the use of an ingested or inserted encapsulated device that delivers a signal to an external data collection device for observation and analysis when a switch sensitive to the ionically conductive environment of the gastrointestinal tract is triggered, thereby indicating that the dose form has been ingested, inserted or otherwise internalized. The data collected in the external data collection device may then be analyzed for management of patient therapy or for clinical study.

The method and system for monitoring and analyzing compliance with a physician-prescribed dosing regimen of the present invention provides an effective and practical response to the problems encountered by the non-compliance of patients to regimens for drugs taken internally. Non-compliance refers to the failure by the patient to take the prescribed dosage at the prescribed time for the prescribed period, resulting in patient under-medication or over-medication. Such non-compliance results in increased cost of medical care, higher complication rates, higher rates of drug-resistance by pathogens, and drug wastage. In a survey of 57 non-compliance studies, failure to comply with the drug regimen ranged from 15% to as high as 95% in all study populations, regardless of medications, patient population characteristics, the drug being delivered, or study methodology. (Greeberg, R. N.: Overview of Patient Compliance with Medication Dosing: A Literature Review, Clin. Therap., 6(5):592-599 [1984].) Reasons for the failure of patients to comply with drug regimens are plentiful and include forgetfulness (30%), other matters taking priority (16%), choosing not to take drug (11%), lack of information (9%) and “emotional factors” (7%). (Osterberg, L., and Blaschke, T.: Compliance to medication, N. Engl. J. Med. 353:5, 490 [2005].)

In the therapeutic setting, accurately measuring and analyzing compliance has a number of important benefits such as enabling the care-giver to warn a patient about the potential for developing a drug resistant infection related to poor compliance to the regimen and enabling the identification of a side effect of a drug related to overdosing. In the clinical drug research stage, accurately measuring and analyzing compliance can lead to a broad range of benefits, including improved statistical reliability of a clinical study, earlier completion of clinical studies, possible identification of side effects, and a determination of the effects of non-compliance as a function of the degree of non-compliance.

Confirmation of drug compliance by way of direct observation by trained persons is effective but impractical in most settings. Confirmation of drug compliance by blood or urine analysis is also not practical beyond the hospital setting.

There have been technical efforts made to overcome the impracticality of direct observation and specimen analysis. These technical efforts have been singularly directed to monitoring dosing compliance. Transdermal detection devices attached to the skin of a patient have been developed which detect ingested drug components through the skin. Such devices can transmit a signal to a remote receiver at an external site such as a healthcare facility as disclosed in, for example, U.S. Pat. No. 6,663,846 and U.S. Published Patent Application No. 2005/0031536. Electronic sensor systems have also been developed which detect ingested drug components in the breath of a patient, such as set forth in U.S. Published Patent Application No. 2004/0081587. Radio Frequency Identification (“RFID”) tags have been incorporated into pills with each tag capable of identifying the type of medication, its dosage, and its lot number by way of a unique code emitted by the tag when interrogated by a corresponding radio frequency reader, as set forth in U.S. Pat. No. 6,366,206. The RFID of the '206 patent can incorporate a biosensor that switches state, for example, by detecting ionic conductivity, in the gastrointestinal tract detects moisture or change in pH to determine whether the pill has dissolved and exposed the RFID tag to the environment of the gastrointestinal system.

Statistical models for drug compliance have also been developed. For example, Gerard et al. in Statistics in Medicine (17, 2313-2333 [1998]) describe a Markov mixed effect model for drug compliance data. Vrijens et al., in Statistics in Medicine (23, 531-544 [2004]), describe a data treatment model for reduced bias and improved precision in pharmacokinetic pharmacodynamic population studies. In European Patent Application No. 0526166 a patient compliance monitoring method using a radio transmitter attached to a medicine container to detect medicine consumption is disclosed. A patient compliance monitoring method based on patient entry of data related to medicine consumption is disclosed in U.S Published Patent Application No. 2002/0143577.

A bar code-based drug dispensing system and database are disclosed in U.S Published Patent Application No. 2003/0055531. In U.S. Published Patent Application No. 2003/0110060, a patient compliance monitoring method that includes interaction with the patient is disclosed. A patient compliance monitoring system which provides the patient with a portable medication dispenser which alerts the patient to take a dose of medication and then gathers compliance data relating to the taking of the medication is set forth in U.S Published Patent Application No. 2004/0133305.

A patient compliance monitoring method employing a pharmacokinetic model to determine if the prescribed dosing regimen should be adjusted is provided in U.S Published Patent Application No. 2004/01193446. The use of a patient compliance monitoring method for use in clinical trials is disclosed in U.S Published Patent Application No. 2004/0243620. A system and method for tracking drug containers is disclosed in U.S Published Patent Application No. 2004/0008123. Finally, a patient compliance monitoring method employing a capsule or pill containing an RFID tag which is responsive to ingestion by a patient is disclosed in U.S Published Patent Application No. 2005/0131281.

Each of the above-described patents and publications provides a contribution to the state of the art with respect to methods and systems for monitoring compliance to a dosing regimen. However, these patents and publications fail to provide a solution to the monitoring of an internal dosing regimen which is fully satisfactory and fail entirely to provide either a method or a system for collecting data generated from a compliance system and for analyzing the collected data in a way that would be useful for either therapy or in the clinical setting.

### SUMMARY OF THE INVENTION

The present invention provides a method and system for monitoring and analyzing compliance with an internal dosing regimen prescribed to be taken in multiple dose forms. In its most fundamental aspect the method of the present invention includes the steps of detecting internalization of a first dose form to generate a first data point, detecting internalization of a second dose form to generate a second data point, and analyzing the first data point and the second data point. The step of analyzing the first and second data points generates a metric of a variety of possible metric types. The first and second dose forms may be two of any plural number of sequentially-internalized dose forms which generate a like number of sequential data points. Subsequent internalizations of dose forms result in at least a like number of data points being generated. Each data point is a time stamp identifier which may include any of a date, a time of the given date, and a serial number of the dose form or may include any of a variety of additional information such as internal body temperature.

To achieve this method the present invention includes a system which includes at least two dose forms, a time stamp identifier operatively associated with each dose form, a receiving device for receiving the time stamp identifier data, and an analyzer for analyzing the received data,

The dose form may include an active ingredient or may be a placebo. The dose form may be orally ingestible or rectally inserted and may be in the form of a capsule, a pill or a tablet. For example, as a capsule containing an effective amount of a drug, the dose form may be embodied in a gelatin-based capsule containing a medicament and a device with a switch sensitive to the conditions in the gastrointestinal tract. The data point may be prepared and emitted by the dose form or may be prepared and emitted by a device external to the body which receives switching information from the dose form. Regardless of the embodiment, the emitted data is received by a temporary data storage device or monitor for receiving and temporarily holding one or more of the emitted data points, which may emit the received data to an intermediate data storage device for temporarily holding the data points received from the temporary data storage device. The system may also include a receiver for receiving and analyzing the emitted data points first emitted.

More particularly and according to one preferred embodiment, the method of the present invention includes using the sensing and signaling device-based system according to the steps of having the dose form containing a sensing and signaling device ingested or inserted by a patient, allowing time for the dissolution of the capsule in the patient's body, activation of the switch in the gastrointestinal tract environment of the patient to create a data point, providing the data point and additional, sequential data points to a temporary storage device, providing the data from the temporary storage device to an intermediate storage device, and providing the data from the intermediate storage device to a caregiver or an analyst for interpretation. The gathered data is then analyzed for verification and interpretation of patient compliance to the prescribed dosing regimen for patient therapy, for clinical investigation, or for both.

According to an alternate embodiment, the method of the present invention includes the steps of having a dose form containing a signaling device ingested or inserted by a patient, allowing time for the dissolution of the dose form in the patient's body, detecting ingestion or insertion of the dose form, recording the detection data in a temporary data storage system to produce a data point, adding the time stamp identifier to the data point to produce an identified data point, storing the identified data point in an intermediate data storage system, repeating the preceding steps for subsequent ingestion or insertions of the dose form, so that the intermediate data storage system contains a plurality of identified data points, intermittently transmitting the plurality of identified data points to a database, and analyzing the plurality of identified data points contained in the database. The analysis of the plurality of identified data points contained within the database is also made for verification and interpretation of patient compliance for either therapeutic purposes, for clinical investigation, or both.

Other features of the invention will become apparent when viewed in light of the detailed description of the preferred embodiment when taken in conjunction with the attached drawings and the appended claims.

### BRIEF DESCRIPTION OF THE DRAWINGS

For a more complete understanding of this invention, reference should now be made to the embodiments illustrated in greater detail in the accompanying drawings and described below by way of examples of the invention wherein:

FIG. 1 is a cross-sectional view of a first embodiment of an example of a dose form as a capsule having signaling capabilities used with the monitoring system of the present invention;

FIG. 2 illustrates the signaling system of the present invention positioned on a user;

FIG. 3 is a flow diagram of the overall method of the present invention illustrating the basic steps of ingestion detection, ingestion data and analysis of the ingestion data;

FIG. 4 is a flow diagram similar to that of FIG. 3 but illustrating additional steps of the method of the present invention;

FIG. 5 is a flow diagram of various methods for carrying out the present invention generally illustrating the steps of simultaneous or substantially simultaneous signal transmission, signal reading, signal storage, and signal interpretation of multiple RFID tags;

FIG. 6 is a graph illustrating findings related to a dosing investigation;

FIG. 7 is a graph illustrating dissolution and on/off switching results of nine capsules;

FIG. 8 is a graph illustrating a first set of in vitro test results for switch testing;

FIG. 9 is a graph illustrating a second set of in vitro test results for switch testing; and

FIG. 10 is a diagrammatic representation of signal strength test results.

### DETAILED DESCRIPTION OF THE PREFERRED EMBODIMENT

In the following figures, the same reference numerals will be used to refer to the same components. In the following description, various operating parameters and components are described for one constructed embodiment. These specific parameters and components are included as examples and are not meant to be limiting.

The method and system of the present invention is directed to monitoring and analyzing compliance with an internal dosing regimen in which the dosing regimen comprises at least a first dose form and a second dose form to be internalized in sequence. The monitored and analyzed compliance may be useful for both aiding the care-giver in optimizing patient therapy and for clinical investigation.

Internalization of the first dose form generates a first data point and internalization of the second dose form generates a second data point which is spaced apart in time from the first data point. The first data point and second data point (or any number of data points generated over time) are then analyzed to generate a metric. The generated and collected data may be analyzed using any manner of a broad variety of analytical methods. Such methods may include, but are not limited to, time series analysis, multivariate analysis, pharmacokinetic regression, pharmacodynamic regression, population comparisons, survival analyses, covariate analyses, single mean analysis, multiple independent group analysis, paired observation analysis, multiple independent groups with paired observation analysis, multiple independent groups with censoring analysis, multiple independent groups with limited recruitment and censoring analysis, single proportion analysis, multiple independent proportions analysis, angular transformation analysis, survival analysis, single correlation analysis, multiple independent correlation analysis, and multiple related correlations analysis.

The generated metric may be any of a variety of metrics which are usable to improve patient compliance with a dosing regime. Specifically, the metric generated may be a compliance metric for a clinical trial, a metric for altering the dose of the medication, a metric for changing the medication, a metric for soliciting communication from the patient, a metric for linking compliance to a warranty of the effectiveness of the medication, a metric for linking compliance to a warranty of the safety of the medication, a metric for determining the efficacy of the medication, a metric for determining the safety of the medication, a metric for establishing trial protocols using the medication, a metric for determining insurability of the medication, and a metric which differentiates a medication for marketing purposes.

System Components

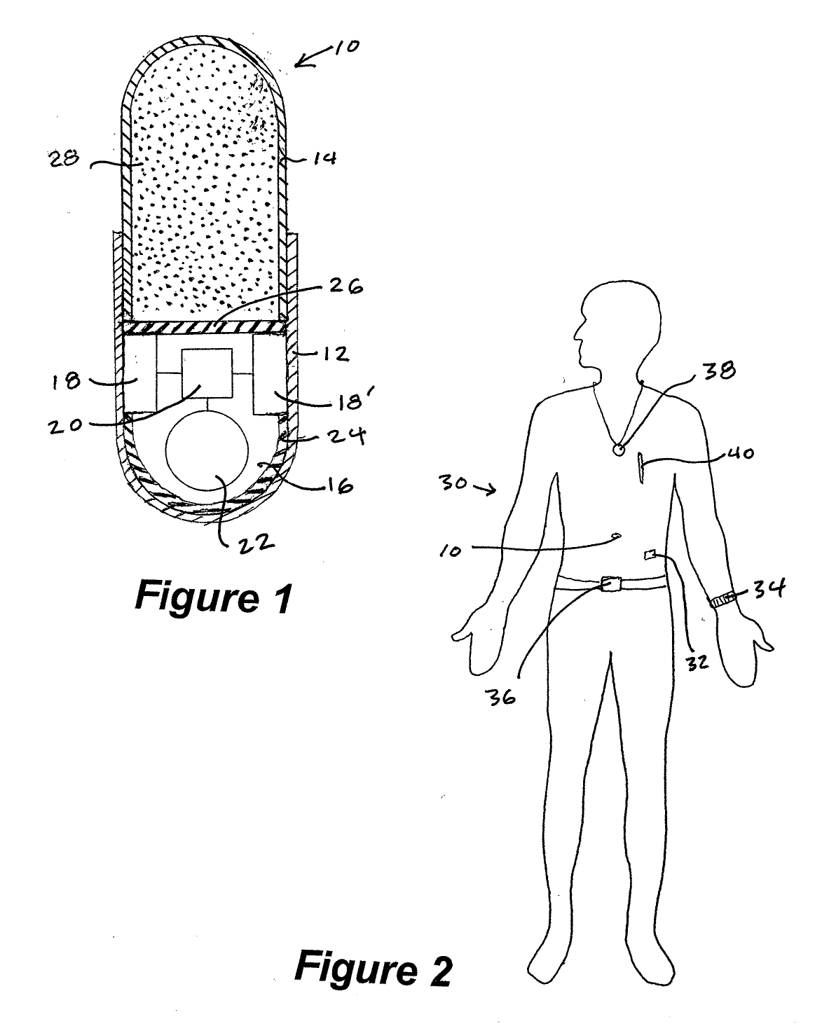

Components of the dose form system of the present invention are illustrate in FIGS. 1 and 2. Referring to FIG. 1, an RFID signaling dose form, generally illustrated as 10, is shown in sectional view. The signaling dose form 10 as illustrated is a capsule, but it is to be understood that other forms of dosing such as tablets and pills may be used as well. The dose form as used herein refers to a dose that includes an active drug ingredient or a may be a placebo.

The dose form 10 is part of a larger system, discussed below, which may find use in both therapeutic practice and in clinical studies. The illustrated capsule is discussed briefly below but the capsule, its structure and its function are set forth in U.S. Patent Application Publication US 2006/0289640, filed on May 18, 2006, and incorporated by reference herein.

As set forth below, the signaling dose form 10 emits a signal to indicate that the dose form 10 has, in fact, been ingested, based upon its having a switch activated by exposure to the gastrointestinal tract. The signal may be emitted in a variety of ways, including, as examples, electromagnetic (e.g., visible light, ultraviolet and infrared radiation, or an RFID signal), magnetic, radioactive, chemical (e.g., a tracer detectable on the breath), fluorescent, acoustic (e.g., ultrasonic or gasified candy-type technology), and biological (e.g., using biomarkers, as from the evolving area of tetramer technology).

The signaling dose form 10 includes an upper gelatin capsule portion 12 and a lower gelatin capsule portion 14. The gelatin capsule portions 12, 14 are of a conventional design and composition.

Housed substantially within the upper gelatin capsule portion 12 is an RFID ionic conductivity-sensing, switching and signal emitting device 16 according to the preferred embodiment. The device 16 may be of any one of several designs and configurations provided that the device 16 is capable of switching on when exposed to the fluids in the gastrointestinal tract and signaling to a monitor (not shown) that the dose form has been ingested. Accordingly, the device 16 as shown is for illustrative purposes only and is not intended as being limiting. The signal from the device 16 can be amplified by a signal amplifier positioned between the device 16 and a signal-receiving and reading device (neither shown).

The device 16 of the dose form 10 may be encoded with a variety of information and may be customized as needed to provide proper assistance to the caregiver or interpreter of the compiled data. By way of non-limiting example, the device 16 may be coded to indicate, among other things, the type of medication, the dose of the medication and the lot and serial numbers of the medication.

The illustrated device 16 includes one or more ionic conductivity sensing elements 18, 18′ operatively associated with a switching/signaling element 20. The switching/signaling element 20 includes a switch which responds to a signal received from the conductivity sensing elements 18, 18′ an antenna which emits a signal to the monitor to provide notification that gastric fluid has been encountered. The device 16 further optionally includes a power source 22. If the device 16 is of the passive type in which power to drive the sensing elements, the switch and the antenna is provided by an incoming radio frequency signal external to the signaling dose form 10, then no on-board power supply is needed. The illustrated device 16, however, is of the active type, in which a power source 22 is provided.

The internal elements of the device 16 are substantially encapsulated in a container 24. The container 24 includes a wall 26 that forms a barrier between its interior space and the interior of the lower gelatin capsule portion 14. Contained within the lower gelatin capsule portion 14 is a quantity of any one of a broad variety of powder, gel or liquid medicaments 28. As set forth above, the dose form 10 may alternatively act as a placebo and have no active ingredient.

Referring to FIG. 2, there is illustrated a signaling and monitoring system 30 of the present invention positioned on a user. The user has swallowed the signaling dose form 10 and it is shown in the approximate area of the user's stomach. However, it should be understood that while the user is illustrated as having taken the signaling dose form 10 orally, the present invention is not limited to oral ingestion. As set forth above in the Brief Description, the utility of the present invention may be extended to use in the colon. Thus the present invention may be used to confirm compliance to a drug regimen for drug absorption in the entire gastrointestinal tract. Accordingly, the capsule ingestion shown in FIG. 2 is intended as being illustrative rather than limiting.

Once ingested, the upper gelatin capsule portion 12 and the lower gelatin capsule portion 14 begin to dissolve. Once the upper gelatin capsule portion 12 has dissolved to the point that one or both of the conductivity sensing elements 18, 18′ contacts the user's gastric juices, the switching/sensing element 20 issues a signal which is received by one or more monitors positioned at various locations on the user's body.

A monitor is regarded as effective for use in the present invention if it can receive and temporarily store data received from the dose form 10 as a signal and relay the received data to a receiving device (not shown) for forwarding, for storage, for real-time interpretation by a caregiver or for other analysis. The data temporarily stored by the monitor is then forwarded on to an intermediate data storage system which may be physically remote from and intermittently communicate with the temporary data storage system embodied in the monitor. Preferably, the intermediate data storage system is contained in a device selected from the group consisting of a cell phone or a personal data assistant, the latter being in the form of a Bluetooth®, a PDA having network capability, Wi-Fi, and the like. The temporary storage device monitor must be worn by the patient during therapy or clinical usage. However, the intermediate storage device may be worn or positioned near the patient on an intermittent basis sufficient so that data from the temporary storage device may be communicated to a receiving data base via the intermediate storage device such that proper care may be rendered.

Preferably, then, the monitor is on or near the patient's body. In circumstances where the patient is not ambulatory, such as in a hospital or in a home-care setting, the monitor may be removably attached to the bed frame or adjacent wall or may be placed bed-side on a table or a night stand. Regardless of the ambulatory disposition of the patient, however, the monitor must be close enough to the patient (to the dose form 10, actually) so as to receive a signal. In the event that the dose form 10 is of the passive device variety, it may be required that the monitor be positioned closer to the patient.

Such monitors may include, but are not limited to, a skin-adhering patch 32, a wrist article (for example, a bracelet, a wrist band, or a wrist watch) 34, a belt buckle 36, a neckpiece (such as a necklace or a pendant) 38, or a pocket device (such as a pen) 40. The monitors 32, 34, 36, 38, 40 are provided for illustrative purposes only and are not intended as being limiting. Other monitoring arrangements may be used including, for example, a storage pack and a hand-held device that could be inserted into a pocket, neither of which is shown.

Procedures for Using the System

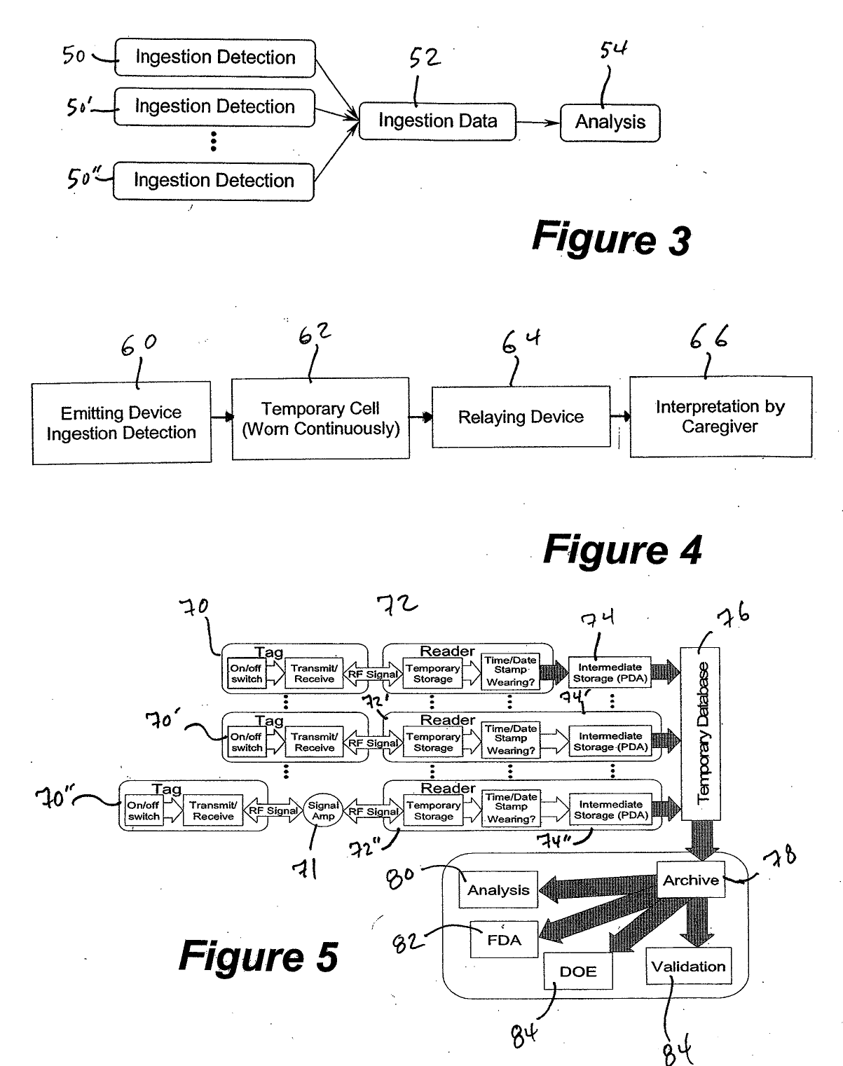

Different procedures for detecting ingestion, generating a set of serial data points for emission to a receiver, collecting the emitted series of data points by the receiver, and interpreting the collected data points according to the method and system of the present invention are set forth in FIGS. 3 through 5. With particular reference to FIG. 3, an overall method of the present invention is illustrated. Confirmation that the patient is complying with the prescribed drug regimen turns on detection of ingestion of the dose form 10, compiling the ingestion data, and analyzing the compiled data. These steps are illustrated in FIG. 3 in which serial detection of sample ingestion steps 50, 50′, 50″ are shown in time sequence. Each ingestion step 50, 50′, 50″ generates a data point that is compiled as ingestion data at step 52. The compiled data of step 52 is then analyzed at step 54. Analysis, as used herein, may in fact be an investigative or reporting step selected from the group consisting of analyzing the plurality of compiled data points, or transferring the plurality of compiled data points for analysis at real time or at a later time. Such transfer can be from any data storage location to another data storage location and by wire, wireless, or optical means.

As set forth in FIG. 4, a flow diagram is shown in which the patient ingests the dose form 10 and the ingestion is detected at step 60. The data generated by the dose form 10 is temporarily gathered in the temporary cell or monitor at step 62. This data is then relayed by a relaying device at step 64 to a receiving device whereby the caregiver can interpret the data at step 66. A time stamp identifier may be added to the series of data points to indicate whether the patient is, in fact, wearing the monitor. The time stamp identifier includes one or more of a serial number, date and time. The time stamp identifier may include other information. It is to be understood that the time stamp identifier may be provided in the dose form 10 and signaled to the monitor at step 61 or may be added by the monitor at step 62.

Referring to FIG. 5, three roughly parallel flow diagrams are illustrated which demonstrate three preferred methodologies according to the present invention. In general, each methodology includes steps in which an internalized tag is switched on in response to a detected change in its environment, the switching event is transmitted to a reader which temporarily stores the emitted signal of the switching event and which generates an information-containing data point based on the switching event, the data point is transferred to an intermediate storage device and then to an archive via a temporary database. The archive then makes the collected data points available for analysis by a caregiver or a data analyzer.

More particularly, a tag provides information in steps 70, 70′, 70″ in which an on-off switch contained in the device 16 responds to conductivity of its environment. Information as to the status of the switch is provided to a transmitter which is part of the device 16. In given circumstances the transmitter may also function as a receiver to receive and respond to an external RF signal. A response to an external RF signal may be, for example, the re-sending of data previously sent. Ordinarily, however, the data being transmitted includes the status of the switch, and thus the status of ingestion. The data sent by the device 16 serially at steps 70, 70′, 70″ is received by a temporary storage and reading device at steps 72, 72′, 72″. It may be that a signal sent by the transmitter of the tag needs to be amplified in which case the signal is boosted at step 71 by a signal amplifier.

The data temporarily stored in the reader at steps 72, 72′, 72″ is time stamped and may be used to confirm whether or not the temporary storage device is being worn by the patient. The transmission of data between steps 70, 70′, 70″ and steps 72, 72′, 72″ may be sent periodically or automatically between the tag and the temporary storage and reading device.

The data gathered and time-stamped at steps 72, 72′, 72″ is then forwarded to an intermediate storage device (such as a PDA) at steps 74, 74′, 74″. In the event that the temporary storage and reading device and the intermediate storage device are separate as between steps 72 and 74, the signal from the reader to the PDA may be intermittent. In the event that the reader and the PDA are single units as illustrated between steps 72′, 72″ and 74′, 74″, then the signals between the reader and the PDA may be periodic or automatic.

Regardless of the arrangement between the reader and the PDA, the data compiled at steps 74, 74′, 74″ by the PDA is communicated to a temporary database at step 76 on an intermittent basis. The information gathered by the temporary database at step 76 representing plural data points is forwarded to an archive at step 78 on an intermittent basis. Finally, the data provided to the archive at step 78 is intermittently forwarded to a variety of end points, including, by way of non-limiting examples, an analyzer for analysis at step 80, the FDA at step 82, the DOE at step 84, or to a researcher for validation at step 84.

Analysis for Patient Therapy

The dose form 10 can provide the caregiver with a broad range of information. The absence of a signal from the dose form 10 indicates that the patient is not complying with the prescribed drug regimen. Incomplete or inconsistent data points from a data point compilation may indicate only marginal compliance to the drug regimen. The caregiver is then able to respond accordingly.

Once the caregiver has interpreted the data for compliance to the prescribed regimen, the patient may be more readily managed. The regimen can be changed and customized for more effective treatment by, for example, changing dosing rates, changing medicines, or modifying the medication on an individual patient or on a patient group basis. This information would also be useful when a drug holiday is established for a patient.

While the compiled data points provide the caregiver or other analyzer with important data as to regimen compliance and the like, it is also possible that the data points generated will be a null set indicating complete non-compliance by the patient. This set of data points also provides valuable information, which would lead the caregiver or data analyzer to conclude that either the patient was not complying with the drug regimen, or that some component of the system of the present invention had failed for technical reasons.

The instant invention also comprises using data analysis results as part of the labeling process for a drug when a manufacturer or seller claims specific performance as a function of an individual's compliance. In addition, the instant invention comprises linking patient compliance to a warranty of effectiveness or safety of a medication or to a confirmation that the patient took the correct and authentic medication.

Analysis for Clinical Investigation

The dose form 10 also has value in clinical studies related to drug typing and efficacy. For clinical trials, for example, the utilization of the compliance data can be used as an input into drug data analyses. In such clinical trials (including in post-market research) the various analyses methods mentioned above may be undertaken. Alternatively or additionally, the instant invention can be used for managing patient dosing during drug trials including working to improve compliance or alternatively soliciting information from the patient as to why compliance is not occurring. One or more than one patient may be monitored using the embodiments of the instant invention depending on the desired analysis. The results of the analysis of the generated data points may be used to produce any one of a number of possible metrics set forth above which will aid in clinical investigation.

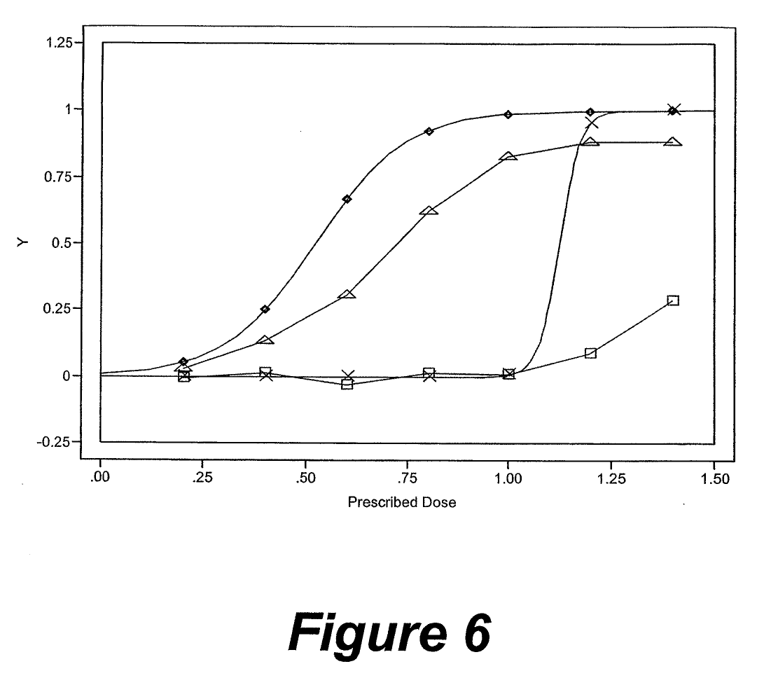

An illustration of how the present method enables, for example, better estimates of safety and efficacy of the medication may be illustrated by way of FIG. 6 in which a graph is shown that discloses the results from a simulated dosing investigation of a population where patients were asked to ingest one dose of medication per day. According to the graph of FIG. 6, x refers to the actual mean side effects, ⋄ refers to actual mean efficacy, □ refers to mean (side effects data) and Δ refers to mean (efficacy data). According to this simulated investigation, each study subgroup was given a different dosing level captured as a multiple of a nominal dose. Furthermore, two clinical endpoints were evaluated during the study. The first measures efficacy and the second the occurrence of a negative side effect. At each dosing level, the subjects may have been randomly noncompliant with respect to their prescribed dosing regimen. For example, if they were 80% compliant, then they did not take 20% of their doses over the trial period. The missed doses are randomly distributed across the trial period. Utilizing the method described in this invention, compliance data were created detecting a plurality of ingestion events for the subjects in a trial.

A probability density distribution was used to model the compliance rate probability for a subject in the simulated trial. Furthermore, sigmoidal equations were used to model the efficacy/side effect dose-response curves, and a normal distribution was used to model the distribution of residuals for each curve. An experimental data set was created via Monte Carlo simulation.

For both effects, a sigmoidal equation was regressed to the data. In the figure, the expected (mean) efficacy and side effect responses are plotted as a function of average dosing, where the lines fit to data (indicated with diamonds and crosses) assume perfect compliance and the lines fit to actual (indicated with triangles and squares) utilize the compliance information from the present invention. More particularly: (1) The line marked with triangles is the model of efficacy derived assuming perfect compliance; (2) the line marked with squares is the model of side effects derived assuming perfect compliance; (3) the line marked with diamonds is the model of efficacy derived using the compliance information from the present invention; and (4) the line marked with crosses is the model of side effects derived using the present invention.

Efficacy and side effects are underestimated in the case where perfect compliance is assumed compared with the case where actual compliance data was used. This leads to prescribing higher than required doses. Moreover, the negative consequences of taking higher doses are underestimated providing a false sense of security. The present invention thus may provide improved estimates of medication safety and efficacy.

Testing of the Active RFID Tracer

To confirm the effectiveness of the active (battery powered) version of the RFID tracer for use in the proposed application testing was undertaken with animal models. Five adult beagle dogs were used. The test animals were 2 to 4 years old, weighing between about 8 and 12 kg. The RFID circuitry and batteries were encapsulated in a medical grade thermoplastic and the full tracer was approximately 8 mm in diameter by 20 mm long. Each of the animals was given a RFID tracer under normal diet conditions. The monitor was suspended in the kennel above but out of reach of the dog.

Each tracer was programmed to transmit a unique serial number and a number indicating the total number of transmissions since switch activation. The time between transmissions was approximately 10 seconds. If the period between transmissions is assumed to be exact, subtracting (1) the number of transmissions times the repeat period from (2) the time of the transmission will give the time the internalization switch was activated. To correct for manufacturing and environmental variations, two different transmissions can be used to calculate the actual transmission period, and this actual value can be used to calculate the time the internalization switch was activated as above.

The time between observed internalization and the calculated internalization switch activation was 1.4 σ 0.6 minutes and the tracer took <24 to 48 hours to pass.

| | Passive RFID | Active RFID |
|---|---|---|
| Average power consumed by | ˜4 Watt | ˜0.1 Watt |
| monitor | | |
| Size of monitor antenna | Disk ˜20 cm in | Rod ˜5 cm long |
| | diameter | |
| Distance from monitor to tracer | ˜15 cm | ˜200 cm |
| Time between observed | 10 σ 5 minutes | 1.4 σ 0.6 |
| internalization and switch | | minutes |
| activation | | |

From the table, one can see that the active tracer: is much more suitable for battery powered and portable operation, has more tolerance for positioning of the monitor on the patient, and has a more accurate and precise estimate of the internalization time.

Testing of the Passive RFID Tracer

To confirm the effectiveness of the passive version of the RFID tracer in the proposed application testing was undertaken with animal models. Four adult beagle dogs were used. The test animals were ˜2 years old, weighing between about 8 and 12 kg. In a first series of tests each of the animals was given a RFID tracer under normal diet conditions. Reading of the tracer signal took 10 σ 5 minutes and the tracer took <24 to 31 hours to pass. In a second series of tests each of the animals was given a tracer following a 16 hour fast. Under these conditions reading of the tracer signal took only 6 σ 1 minutes but the tracer took 24 to 48 hours to pass.

Dissolution Testing

Dissolution of the RFID tracer model of the present invention was tested using an encapsulated pFET transistor switch powered by a small battery. The tracer model used an LED device in lieu of an RFID. An 00-sized gelatin capsule having a sucrose backfill and based on an acetaminophen model of 150 mg dose was introduced into a 900 ml tank of simulated gastric fluid maintained at 37° C. Testing was done at both pH 1.3 and pH 5.8.

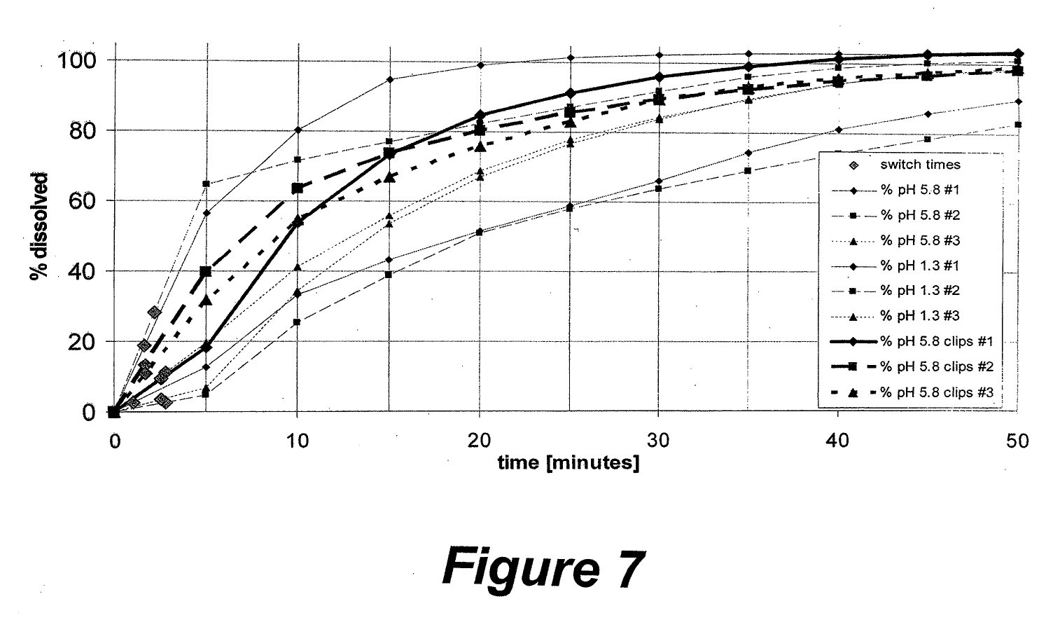

The results of the dissolution testing appear in FIG. 7 in which the percentage of dissolution is on the Y-axis and the time of dissolution (in minutes) is set forth on the X-axis. Three test RFID tracer models were evaluated. Summarizing FIG. 7, the test data support an overall time until switching of 2.1 σ 0.6 minutes with an overall time to 50% dissolution of 20 σ 14 minutes. The test data also demonstrate an overall %-dissolution at switching time of 11 σ 8% while the use of clips resulted in an overall %-dissolution at switching time of 11 σ 2%.

Testing to Establish Switch Characteristics

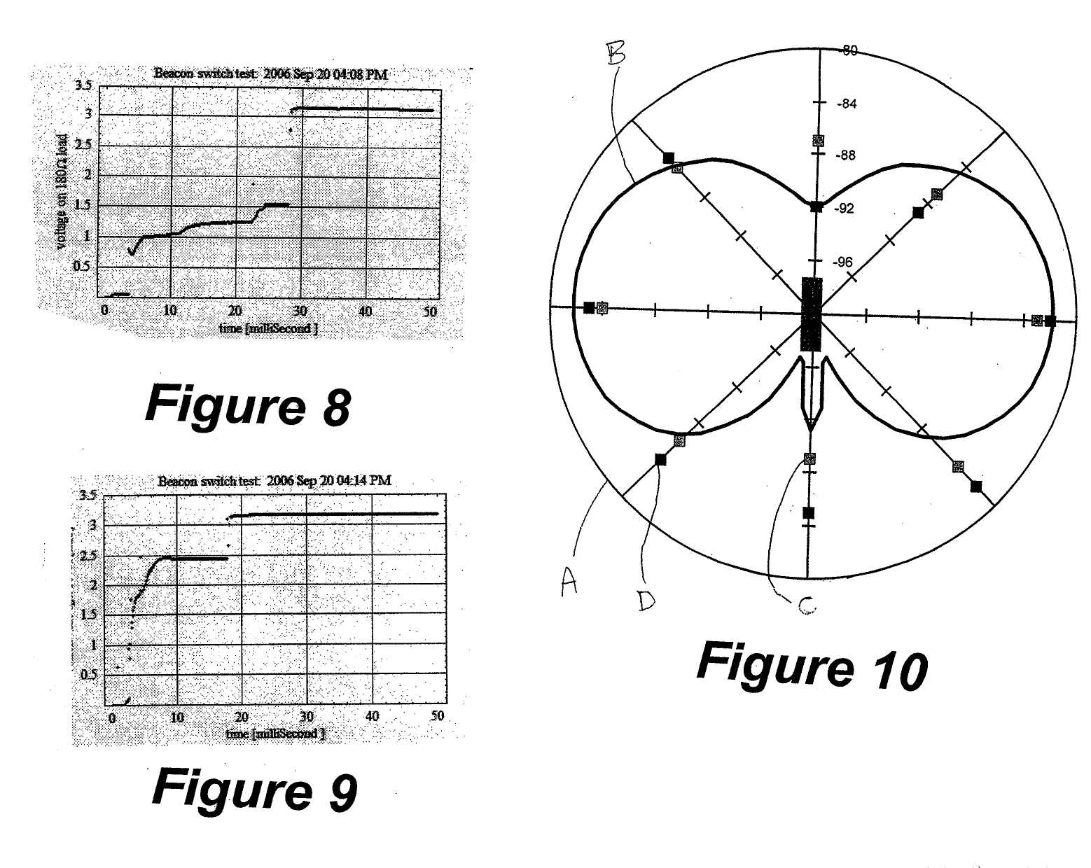

The effectiveness of the proposed switch was tested using USP dissolution test instrumentation. To make this assessment a current through a load resistor with 3 volts across the switch of the RFID tracer model discussed above was measured. This current was measured every three milliseconds 0beginning from the time the capsule was immersed in the simulated gastric fluid until one minute after switch actuation. For these tests the simulated gastric fluid was maintained at 37° C. at a pH level of 1.2. The results of this analysis are set forth in FIGS. 8 and 9 in which time 0 (along the X-axis) is about 2 minutes after immersion. Voltage (on 180 Ω load) is shown on the Y-axis.

With reference to FIG. 8, at a little over two minutes following immersion (at about 5.0 milliseconds) a signal is detected which gradually increases in strength as the capsule becomes more completely dissolved until about 28.0 milliseconds after immersion when a signal of about 3.1 is detected.

With reference to FIG. 9, at about 4.0 milliseconds after time 0 a signal is detected. As the capsule is more completely dissolved in the simulated gastric fluid signal strength increases, this time more dramatically than the test of FIG. 8, to about 2.5 after only about 7.5 milliseconds after time 0. The voltage levels off at about 2.5 until about 18 milliseconds after time 0 when the voltage climbs to about 3.1 and holds at this level.

Radio Frequency Testing

To determine the strength of the signal emitted from the RFID tracer model and to account for various organs, bones and connective tissue, a series of radio frequency tests were undertaken. Human gut was imitated using a Specific Absorption Rate (SAR) simulation composition. The composition was composed of water (52.4%), salt (1.4%), sugar (45%), HEC (hydroxyethyl cellulose, a thickener; 1.0%), and a bactericide (0.1%). The composition was placed in a testing tank.

An antenna arrangement consistent with that used in the RFID tracer model was encased in a thermoset resin to form a sealed unit. A monitor was placed near the testing tank. The sealed unit was submerged into the simulated human gut composition in the testing tank to determine (1) signal strength vs. signal distance, (2) power draw, and (3) signal direction.

Signal strength testing generated the following results:

| | Signal strength |
|---|---|
| Description | (in nanowatts) |
| Minimum usable power | 0.0001 |
| Measurement of dipole in tank | 5.0 |
| Measurement of dipole just outside | 0.1 |
| tank | |
| Measurement of dipole 70 cm from | 0.01 |
| tank wall | |

With respect to (1) signal strength vs. signal distance, the test data support a conclusion that the signal strength readily meets the objectives for signal robustness, including length of time to transmit the signal and low error rates on the detected signal. Additional tests show that even at 2 meters from a test tank the emitted signal was well received by the monitor.

With respect to (2) power draw, the test data also support a conclusion that only a relatively small battery is needed to generate a signal that can be regarded as adequate.

With respect to (3) signal direction, FIG. 10 illustrates signal strength (in dBm) of the test unit relative to the immersion tank. The outer circle A represents the outer wall of the testing tank. The line B defining the two joined partial circles represents the computation line. Signal strength measured in air is represented by the diamonds C while the signal strength measured with an interfering human arm is represented by the diamonds D. The test data support several conclusions, including location of the RFID tracer within the stomach will not represent a significant barrier (if at all) to signal detection, signal transmission is fast enough so that movement within the stomach will not interfere with signal detection, and the presence of a human arm does not interfere with signal measurement in any significant way.

### EXAMPLES

The following examples are provided by way of explanation of the present invention and are not intended on being limiting.

### Example 1

A passive RFID tag coded with medication type, dose and lot number is contained in capsules of the medication so that when a patient ingests the capsule, the RFID tag is dispersed into the digestive tract and is turned on by a moisture-sensitive switch associated with the RFID tag. The patient wears an electronic patch adhering to his skin that comprises an RFID reader and a temporary data storage device. When ingestion is detected, a data point is generated related to the medication type, dose and lot number which data point is time and date stamped and placed in temporary data storage. Intermittently, the electronic patch communicates with an intermediate data storage device in the form of a personal data assistant (PDA). Intermittently, the PDA communicates the stored data to a temporary database. The data in the temporary database is analyzed to produce a metric. The generated metric may be any of a variety of metrics which are usable to improve patient compliance with a dosing regimen.

### Example 2

An active RFID tag coded with medication type, dose and lot number is contained in capsules of the medication so that when a patient ingests the capsule, the RFID tag is dispersed into the digestive tract and is turned on by an electrical conductivity sensitive switch associated with the RFID tag. The patient is wearing an electronic patch adhering to his skin that comprises an RFID reader, a temporary data storage device and an intermediate data storage device. When ingestion is detected, a data point is generated related to the medication type, dose and lot number which data point is time and date stamped and placed in temporary data storage. Periodically or automatically, the temporary data storage device communicates with an intermediate data storage device. Intermittently, the intermediate data storage device communicates the stored data to a temporary database. The data in the temporary database is analyzed to produce a metric. The generated metric may be any of a variety of metrics which are usable to improve patient compliance with a dosing regimen.

### Example 3

An active RFID tag coded with medication type, dose and lot number is contained in tablets of the medication so that when a patient ingests the capsule, the RFID tag is dispersed into the digestive tract and is turned on by an electrical conductivity sensitive switch associated with the RFID tag. The patient's environment comprises a base station comprising an RFID reader, a temporary data storage device and an intermediate data storage device. The patient is wearing an electronic patch comprising a transceiver which receives signals from the RFID tag and then transmits an amplified signal to the base station. When ingestion is detected, a data point is generated related to the medication type, dose and lot number, which data point is time and date stamped and placed in the temporary data storage of the base station. Periodically or automatically, the temporary data storage device communicates with an intermediate data storage device. Intermittently, the intermediate data storage device communicates the stored data to a temporary database. The data in the temporary database is analyzed to produce a metric. The generated metric may be any of a variety of metrics which are usable to improve patient compliance with a dosing regimen.

### Example 4

A patient ingests a drug capsule containing a dose form and a highly crystalline and frangible water-permeable material, thereby fracturing the highly crystalline and frangible water-dispersible material to produce sound waves. The sound waves are detected by a sound sensor system incorporated into a patch worn on the patient's abdomen, the sound sensor system including a piezo microphone connected to electronics comprising an amplifier and a microprocessor/data logger. When ingestion is detected, a data point is generated, which data point is time and date stamped and placed in the temporary data storage. Periodically or automatically, the temporary data storage device communicates with an intermediate data storage device. Intermittently, the intermediate data storage device communicates the stored data to a temporary database. The data in the temporary database is analyzed to produce a metric. The generated metric may be any of a variety of metrics which are usable to improve patient compliance with a dosing regimen.

### Example 5

A patient ingests a drug capsule containing a dose form and a magnet. When the patient swallows the drug capsule, a magnetic field detector positioned around the neck of the patient detects the ingestion of the drug capsule and generates a data point, which data point is time and date stamped and placed in the temporary data storage. Periodically or automatically, the temporary data storage device communicates with an intermediate data storage device. Intermittently, the intermediate data storage device communicates the stored data to a temporary database. The data in the temporary database is analyzed to produce a metric. The generated metric may be any of a variety of metrics which are usable to improve patient compliance with a dosing regimen.

### Example 6

A patient ingests a drug tablet containing a dose form and a fluorophore (rhodaminine 800). The fluorophore enters the bloodstream and is detected by the transdermal detection device detailed in WO0037114. When ingestion is detected, a data point is generated, which data point is time and date stamped and placed in the temporary data storage. Periodically or automatically, the temporary data storage device communicates with an intermediate data storage device. Intermittently, the intermediate data storage device communicates the stored data to the temporary database. The data in the temporary database is analyzed to produce a metric. The generated metric may be any of a variety of metrics which are usable to improve patient compliance with a dosing regimen.

The foregoing discussion discloses and describes an exemplary embodiment of the present invention. One skilled in the art will readily recognize from such discussion, and from the accompanying drawings and claims that various changes, modifications and variations can be made therein without departing from the true spirit and fair scope of the invention as defined by the following claims.

What is claimed is:

1. A method for monitoring compliance with a dosing regimen comprising the steps of:
detecting internalization of a first dose form to generate a first data point;
detecting internalization of a second dose form to generate a second data point; and
analyzing said first data point and said second data point.

2. The method for monitoring compliance with a dosing regimen of claim 1 wherein said step of analyzing said first data point and said second data point generates a metric, said metric being selected from the group consisting of a compliance metric for a clinical trial, a metric for altering the dose of the medication, a metric for changing the medication, a metric for soliciting communication from the patient, a metric for linking compliance to a warranty of the effectiveness of the medication, a metric for linking compliance to a warranty of the safety of the medication, a metric for determining the efficacy of the medication, a metric for determining the safety of the medication, a metric for establishing trial protocols using the medication, a metric for determining insurability of the medication, and a metric which differentiates a medication for marketing purposes.

3. The method for monitoring compliance with a dosing regimen of claim 1 wherein said step of analyzing said first data point and said second data point is performed using one or more of time series analysis, multivariate analysis, pharmacokinetic regression, pharmacodynamic regression, population comparisons, survival analyses, covariate analyses, single mean analysis, multiple independent group analysis, paired observation analysis, multiple independent groups with paired observation analysis, multiple independent groups with censoring analysis, multiple independent groups with limited recruitment and censoring analysis, single proportion analysis, multiple independent proportions analysis, angular transformation analysis, survival analysis, single correlation analysis, multiple independent correlation analysis, and multiple related correlations analysis.

4. The method for monitoring compliance with a dosing regimen of claim 1 wherein said first data point is a first time stamp identifier assigned to said first dose form and said second data point is a second time stamp identifier assigned to said second dose form.

5. The method for monitoring compliance with a dosing regimen of claim 1 wherein said first data point is a first serial number assigned to said first dose form and said second data point is a second serial number assigned to said second dose form.

6. The method for monitoring compliance with a dosing regimen of claim 1 wherein said first data point and said second data point are produced in a temporary data storage system in which a time stamp identifier is added to said first data point to produce an identified first data point and in which a time stamp identifier is added to said second data point to produce an identified second data point.

7. The method for monitoring compliance with a dosing regimen of claim 6 wherein said identified first data point and said identified second data point are transmitted to and stored in an intermediate data storage system.

8. The method for monitoring compliance with a dosing regimen of claim 7 wherein said identified first data point and said identified second data point are transmitted intermittently from said intermediate data storage system to a database.

9. The method for monitoring compliance with a dosing regimen of claim 1 in which detection of internalization of said doses is accomplished using a material associated with said dose forms which permits detection by a method selected from the group consisting of electromagnetic detection, magnetic detection, radioactive detection, fluorescent detection, acoustic detection and chemical detection.

10. A method for monitoring compliance with a dosing regimen involving a dose form to be taken internally in the body of a user, the method comprising the steps of:
preparing a first dose form comprising a detector;
preparing a second dose form comprising a detector;
placing said first dose form into the body of the user;
detecting the environment of the body adjacent to said first dose form;
generating a first data point in response to the detection of the environment inside the body by said first dose form;
placing said second dose form into the body of the user;
detecting the environment of the body adjacent to said second dose form;
generating a second data point in response to the detection of the environment inside the body by said second dose form; and
analyzing said first data point and said second data point.

11. The method for monitoring compliance with a dosing regimen of claim 10 wherein said step of analyzing said first data point and said second data point generates a metric, said metric being selected from the group consisting of a compliance metric for a clinical trial, a metric for altering the dose of the medication, a metric for changing the medication, a metric for soliciting communication from the patient, a metric for linking compliance to a warranty of the effectiveness of the medication, a metric for linking compliance to a warranty of the safety of the medication, a metric for determining the efficacy of the medication, a metric for determining the safety of the medication, a metric for establishing trial protocols using the medication, a metric for determining insurability of the medication, and a metric which differentiates a medication for marketing purposes.

12. The method for monitoring compliance with a dosing regimen of claim 10 wherein said step of analyzing is performed using one or more of time series analysis, multivariate analysis, pharmacokinetic regression, pharmacodynamic regression, population comparisons, survival analyses, covariate analyses, single mean analysis, multiple independent group analysis, paired observation analysis, multiple independent groups with paired observation analysis, multiple independent groups with censoring analysis, multiple independent groups with limited recruitment and censoring analysis, single proportion analysis, multiple independent proportions analysis, angular transformation analysis, survival analysis, single correlation analysis, multiple independent correlation analysis, and multiple related correlations analysis.

13. The method for monitoring compliance with a dosing regimen of claim 10 wherein said first data point is a first time stamp identifier assigned to said first dose form and said second data point is a second time stamp identifier assigned to said second dose form.

14. The method for monitoring compliance with a dosing regimen of claim 10 wherein said first data point is a first serial number assigned to said first dose form and said second data point is a second serial number assigned to said second dose form.

15. The method for monitoring compliance with a dosing regimen of claim 10 wherein said first data point and said second data point are produced in a temporary data storage system in which a time stamp identifier is added to said first data point to produce an identified first data point and in which a time stamp identifier is added to said second data point to produce an identified second data point.

16. The method for monitoring compliance with a dosing regimen of claim 15 wherein said identified first data point and said identified second data point are transmitted to and stored in an intermediate data storage system.

17. The method for monitoring compliance with a dosing regimen of claim 16 wherein said identified first data point and said identified second data point are transmitted intermittently from said intermediate data storage system to a database.

18. The method for monitoring compliance with a dosing regimen of claim 10 in which detection of internalization of said doses is accomplished using a material associated with said dose forms which permits detection by a method selected from the group consisting of electromagnetic detection, magnetic detection, radioactive detection, fluorescent detection, acoustic detection and chemical detection.

19. A system for monitoring compliance to a dosing regimen by a user comprising:
a first dose form for placement into the body of the user, said first dose form including a device for detecting placement into the body;
a second dose form for placement into the body of the user, said second dose form including a device for detecting placement into the body;
a producing device for producing a first data point in response to the detection of placement of said first dose form into the body of the user;
a producing device for producing a second data point in response to the detection of placement of said second dose form into the body of the user;
a receiving device for receiving said first data point and said second data point; and
an analyzer for analyzing said first data point and said second data point.

20. The system for monitoring compliance to a dosing regimen of claim 19 wherein said producing device for producing said first data point produces a time stamp identifier and said producing device for producing said second data point produces a time stamp identifier.
